(19) United States
(12) Patent Application Publication
(10) Pub. No.: US 2020/0345768 A1
(43) Pub. Date: Nov. 5, 2020

(54) ZINC PICOLINATE, MAGNESIUM PICOLINATE AND SELENIUM METHIONINE COMPOSITIONS AND METHODS OF USE

(71) Applicant: NUTRITION 21, LLC, Purchase, NY (US)

(72) Inventors: James R. Komorowski, Trumbull, CT (US); Sara Perez Ojalvo, New York, NY (US); Sarah Sylla, Purchase, NY (US)

(21) Appl. No.: 16/865,263

(22) Filed: May 1, 2020

Related U.S. Application Data
(60) Provisional application No. 62/841,336, filed on May 1, 2019.

(51) Int. Cl. A61K 33/30; A61K 33/06; A61K 33/04; A61K 31/4402; A61K 31/198; A61P 15/10; A61P 19/08

(52) U.S. Cl. CPC A61K 33/30; A61K 33/06; A61K 33/04; A61P 19/08; A61K 31/198; A61P 15/10; A61K 31/4402

(57) ABSTRACT

The present application relates to zinc picolinate, magnesium picolinate and selenium methionine compositions and methods of use. The methods and compositions disclosed herein are particularly useful for optimizing antioxidant and hormone levels and improving bone mineral density.

### CROSS-REFERENCE TO RELATED APPLICATIONS

This Application claims the benefit of U.S. Provisional Application 62/841,336 filed on May 1, 2019, the disclosure of which is incorporated herein by reference.

### FIELD OF THE INVENTION

The present invention relates generally to the diet, and more specifically to optimizing absorption of various compounds and compositions, and optimizing use of various compounds and compositions in physiological pathways.

### BACKGROUND OF THE INVENTION

The present application relates to zinc picolinate (ZnPic), magnesium picolinate (MgPic) and selenium methionine (SeMet) compositions (collectively known as ZMS) and methods of use. The methods and compositions disclosed herein are particularly useful for optimizing mineral absorption, antioxidant and hormone levels in mammals.

Magnesium (Mg), selenium (Se), and zinc (Zn) are essential trace elements with antioxidant properties that are important in multiple metabolic processes in organisms. For example, magnesium plays an important role in a wide variety of critical cellular processes, including oxidative phosphorylation, glycolysis, cellular respiration and protein synthesis (Bardgett et al., 2005, Xu et al., 2014). Symptoms of magnesium deficiency include tetany, seizures, arrhythmias and neuromuscular irritability. Low magnesium levels may also contribute to hypocalcemia and hypokalemia. There is also evidence that magnesium deficiency is involved in many age-related phenotypes including sarcopenia and metabolic syndrome (Barbagallo & Dominguez, 2010). Magnesium supplementation could exert beneficial effects on fasting and postprandial glucose levels and insulin sensitivity, including a significant reduction in plasma cholesterol and LDL cholesterol, and an increase in HDL cholesterol in patients with type 2 DM. In addition, magnesium supplementation has been associated with a better anabolic hormonal profile. In a generally well-nourished population of middle-aged to elderly men, plasma IGF-1 levels tended to increase with higher intake of protein and minerals, including magnesium (Giovannucci et al., 2003). Magnesium is an abundant mineral that occurs naturally both in the body and in many foods but compositions disclosed herein do not occur naturally and, as further described herein, prior methods of synthesis have failed to produce adequate compositions. Magnesium is an essential co-factor in many physiological processes, including energy production, protein synthesis and transport, cell signaling, and homeostatic regulation. Indeed, magnesium exhibits bioactivity in all cells and tissues of the body.

Dietary magnesium may be obtained from milk, oatmeal, fruits, vegetables, nuts, and seeds. Uptake of magnesium occurs primarily in the small intestine and secondarily in the large intestine. Most absorption is paracellular, although some is transcellular, through the TRPM channels. Magnesium absorption in the gastrointestinal tract is normally between 25-75%.

Despite its abundance in nature, magnesium deficiency is a widespread heath concern across the United States and globally. According to recent estimates, more than half of Americans are magnesium deficient. Magnesium deficiency is most common amongst individuals with elevated excretion of nutrients from the gastrointestinal tract (e.g., diarrhea). Thus, individuals having, for example, Crohn's disease, celiac disease, type II diabetes, and alcoholism frequently exhibit magnesium deficiency.

Magnesium deficiency and/or impaired magnesium utilization can have serious physiological consequence. For example, decreased magnesium levels in the brain have been associated with migraines, stroke, depression, Parkinson's, and other neurological disorders. Decreased magnesium levels in the lungs are associated with asthma and COPD. Decreased magnesium levels in the heart are associated with coronary artery disease, myocardial infarction, and abnormal blood flow. A decreased magnesium level in muscle is associated with cramps. A decreased magnesium level in bone is associated with osteoporosis.

Zinc has various biological benefits and supplementation has been shown to prevent increases in transaminases, reduce liver fibrosis and triglyceride levels, and improve the long-term outcome of chronic hepatitis C patients. Zinc is known to be involved in synthesis, storage, and release of insulin. Plasma zinc levels are lower in obese individuals and studies have revealed that zinc has beneficial effects on insulin resistance, glucose, and lipid profile in patients with diabetes or metabolic syndrome (Farvid et al., 2004; Kadhim et al., 2006).

Selenium is an important component of several enzymes like thioredoxin reductase, glutathione peroxidase (GPx), selenoprotein P and iodothyronine 5′-diodinase that are required in regulating gene expression, the antioxidant system, and proliferation. (Deepmala et al., 2013). Magnesium and zinc supplementation increase testosterone levels in strength-trained, competitive athletes (Brilla & Conte, 2000) and 4 weeks of magnesium supplementation increases testosterone levels in healthy, sedentary and young male athletes. It is well known that mineral chelates with organic compounds have lower toxicity and higher bioavailability than inorganic forms. A number of studies dealing with the effects of different Mg chelates [nicotinate, propionate, methionine] on animals are available. In 2000, a placebo-controlled, double-blind study showed that the use of the zinc-containing nutritional supplement ZMA by semiprofessional athletes resulted in an increase of plasma testosterone levels of approximately 30% and significantly improved muscle strength when compared to control athletes (Brilla and Conte, 2000). According to the manufacturer's information, the ZMA supplement contained zinc (30 mg per recommended dose of three capsules, present as monomethionine and aspartate), magnesium (450 mg, as aspartate) and vitamin B6 (10.5 mg, as hydrochloride) (Koehler et al., 2009). Magnesium picolinate is a newly developed chelate. In this study, we will compare the effects of ZMS [combination of magnesium picolinate (MgPic), zinc picolinate (ZnPic) and selenomethionine (SeMet)] and ZMA [combination of ZnAspartate (ZnAsp) and ZnMonomethionine (ZnMet), MgAspartate (MgAsp) and Vitamin B6] on visceral fat, biochemical parameters, leptin, anabolic hormones including testosterone (free and total) and estradiol, serum minerals (magnesium, zinc and selenium), IGF-1, FSH, LH, SHBG, BDNF, MDA, antioxidant enzymes, absorption of minerals, retina VEGF, iNOS, ICAM Nrf2, bone mineral density, osteocalcin, bone minerals and grip strength in rats fed with a high-fat diet (HFD).

### SUMMARY OF THE INVENTION

In some embodiments, the present invention comprises nutritional and therapeutic compositions that are useful for optimizing mineral absorption in mammals. In some embodiments, the present invention comprises nutritional and therapeutic compositions that are useful for optimizing antioxidant levels in mammals. In some embodiments, the present invention comprises nutritional and therapeutic compositions that are useful for optimizing hormone levels in mammals. In some embodiments, the present invention comprises nutritional and therapeutic compositions that are useful for optimizing anabolic hormone levels in mammals. In some embodiments, the present invention comprises nutritional and therapeutic compositions that comprise zinc picolinate, magnesium picolinate and selenium methionine.

In some embodiments, the present invention comprises nutritional and therapeutic compositions that comprise zinc picolinate, magnesium picolinate and selenium methionine and a pharmaceutically acceptable vehicle, carrier, or diluent. In some embodiments, the composition is a solid composition. Other features, advantages, and embodiments of the invention will be apparent to those of ordinary skill in the art from the following description, examples, and appended claims.

In some embodiments, the composition comprises a sustained-release matrix. In some embodiments, the composition is enteric coated. In some embodiments, the composition comprises between about 10 μg to about 1,000 mg of zinc picolinate, between about 10 μg to about 10,000 mg magnesium picolinate and between about 10 μg to about 1,000 mg of selenium methionine.

### BRIEF DESCRIPTION OF THE DRAWINGS

FIG. 1 is graph depicting the effects of ZMS on free testosterone levels.

### DETAILED DESCRIPTION

The terminology used in the description presented herein is not intended to be interpreted in any limited or restrictive manner, simply because it is being utilized in conjunction with a detailed description of certain specific embodiments described herein. Furthermore, embodiments described herein can include several novel features, no single one of which is solely responsible for its desirable attributes or which is essential to practicing the embodiments described herein.

As used in the claims below and throughout this disclosure, the phrase “consisting essentially of” is meant to include any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and can or cannot be present depending upon whether or not they affect the activity or action of the listed elements.

As used herein, a composition that “substantially” comprises a compound means that the composition contains more than about 80% by weight, more preferably more than about 90% by weight, even more preferably more than about 95% by weight, and most preferably more than about 98% by weight of the compound.

The term “about,” unless otherwise stated explicitly herein, means ±20%. For instance about 100 means 80 to 120, about 5 means 4 to 6, about 0.3 means 0.24 to 0.36, and about 60% means 48% to 72% (not 40% to 80%).

The term “pharmaceutical formulation” refers to preparations which are in such a form as to permit the biological activity of the active ingredients to be effective, and, therefore may be administered to a subject for therapeutic use.

A “therapeutically effective amount” as used herein includes within its meaning a non-toxic but sufficient amount of a compound active ingredient or composition comprising the same for use in the embodiments disclosed herein to provide the desired therapeutic effect. Similarly “an amount effective to” as used herein includes within its meaning a non-toxic but sufficient amount of a compound active ingredient or composition comprising the same to provide the desired effect. The exact amount of the active ingredient disclosed herein required will vary from subject to subject depending on factors such as the species being treated, the age and general condition of the subject, the severity of the condition being treated, the particular agent being administered, the weight of the subject, and the mode of administration and so forth. Thus, it is not possible to specify an exact “effective amount.” However, for any given case, an appropriate “effective amount” may be determined by one of ordinary skill in the art using only routine methods. In some aspects, a therapeutically effective amount may include a dosing regimen. For example, a therapeutically effective amount may include about 1 mg of a composition orally consumed each day for fourteen consecutive days. In some aspects, a therapeutically effective amount may include about 1 mg of a composition orally consumed each day for thirty consecutive days. It should also be noted that the dosage of ingredients can be configured for a desired amount of elemental magnesium, zinc or selenium. For example, one gram of MgPic would contain 9% elemental Mg by weight (i.e., 0.09 g of Mg in 1 g of MgPic).

In addition, the appropriate dosage of the compositions will depend, for example, on the condition to be treated, the severity and course of the condition, whether the composition is administered for preventive or therapeutic purposes, previous therapy, the patient's clinical history and response to the composition, the type of composition used, and the discretion of the attending physician. The composition is suitably administered to the patient at one time or over a series of treatments and may be administered to the patient at any time from diagnosis onwards. The composition may be administered as the sole treatment or in conjunction with other drugs or therapies useful in treating the condition in question.

By way of example, a “therapeutically effective amount” of the composition disclosed herein can be, for example, 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 80 μg/kg 0, 850 μg/kg, 900 μg/kg, 1 mg/kg, 1.5 mg.kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, or more, or any fraction in between of the composition.

Accordingly, in some embodiments, the dose of the composition disclosed herein can be about 10 μg to about 10 g, preferably per day. For example, the amount of the composition can be 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 g, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 15 g, 20, g, 30 g, 40 g, 50 g or more, or any range or amount in between any two of the preceding values. For example, a dose may comprise between about 10 μg and 100 μg, about 1,000 μg to about 10,000 μg, about 10 mg and 100 mg, about 100 mg and about 1000 mg, about 50 mg to about 1000 mg, about 500 and about 1000 mg, about 1 g to about 50 g, and ranges therebetween. The exemplary therapeutically effective amounts listed above, can, in some embodiments be administered in the methods described elsewhere herein on an hourly basis, e.g., every one, two, three, four, five, six, seven, eight, nine, ten, eleven, twelve, thirteen, fourteen, fifteen, sixteen, seventeen, eighteen, nineteen, twenty, twenty-one, twenty-two, twenty-three hours, or any interval in between, or on a daily basis, every two days, every three days, every four days, every five days, every six days, every week, every eight days, every nine days, every ten days, every two weeks, every month, or more or less frequently, as needed to achieve the desired therapeutic effect.

As used herein, the term “excipient material” refers to any compound that is part of a formulation that is not an active ingredient, i.e., one that has no relevant biological activity, and which is added to the formulation to provide specific characteristics to the dosage form, including by way of example, providing protection to the active ingredient from chemical degradation, facilitating release of a tablet or caplet from equipment in which it is formed, and so forth.

For the purpose of this disclosure, a warm-blooded animal is a member of the animal kingdom which includes but is not limited to mammals and birds. The most preferred mammal of this application is a human.

As used herein, the term “nutraceutical” and “pharmaceutical” can be used interchangeably and distinctly, and their meaning will be clear to the skilled artisan in consideration of the context in which they are used. For example, a “pharmaceutically acceptable solvent” can be interpreted to include a “nutraceutically acceptable solvent” but not necessarily vice versa. The compositions described herein may be referred to as “nutraceuticals” or “dietary supplements” and these terms may be used interchangeably. “Pharmaceutical” can encompass “nutraceutical” and “nutraceutical” can encompass a “dietary supplement” but neither a “dietary supplement” nor a “nutraceutical” can be a “pharmaceutical.” When describing nutraceuticals and dietary supplements, these terms are to be interpreted in the manner that would be given to them by the skilled artisan and in consideration of the guidelines of the U.S. Food and Drug Administration. Nutraceutical and dietary supplement compositions described herein may also include ingredients or components that are defined as generally recognized as safe (GRAS).

To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The administration of one or more of the compositions disclosed herein can be by any of the methods of administration described herein or by delivery methods known by one of skill in the art. The compositions may be administered orally, through parenteral nutrition, e.g., feeding tube, intravenously, or topically, and through other known means.

For oral administration, the compositions disclosed herein can be provided as a tablet, aqueous or oil suspension, dispersible powder or granule, emulsion, hard or soft capsule, syrup, elixir, or beverage. Solid dosage forms such as tablets and capsules may comprise an enteric coating. Compositions intended for oral use can be prepared according to any method known in the art for the manufacture of pharmaceutically acceptable compositions and such compositions may include one or more of the following agents: sweeteners, flavoring agents, coloring agents, coatings, and preservatives. The sweetening and flavoring agents will increase the palatability of the preparation. Tablets containing the complexes in admixture with non-toxic pharmaceutically acceptable excipients suitable for tablet manufacture are acceptable. Pharmaceutically acceptable vehicles such as excipients are compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets can be uncoated or can be coated by known techniques to delay disintegration and absorption in the gastrointestinal tract and thereby provide a sustained action over a longer period of time. For example, a time delay material such as glyceryl monostearate or glyceryl distearate alone or with a wax can be employed.

Formulations for oral use can also be presented as hard gelatin or non-gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil. Aqueous suspensions can contain the complex of the invention in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions can be formulated by suspending the active ingredient in a vegetable oil, such as arachis oil, olive oil, sesame oil or coconut oil, or in a mineral oil such as liquid paraffin. The oil suspension can contain a thickening agent, such as beeswax, hard paraffin or cetyl alcohol. Sweetening agents, such as those set forth above, and flavoring agents can be added to provide a palatable oral preparation. These compositions can be preserved by an added antioxidant such as ascorbic acid. Dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, can also be present.

Syrups and elixirs can be formulated with sweetening agents, such as glycerol, sorbitol or sucrose. Such formulations can also contain a demulcent, a preservative, a flavoring or a coloring agent.

The composition for parenteral administration can be in the form of a sterile injectable preparation, such as a sterile injectable aqueous or oleaginous suspension. This suspension can be formulated according to methods well known in the art using suitable dispersing or wetting agents and suspending agents. The sterile injectable preparation can also be a sterile injectable solution or suspension in a non-toxic parenterally-acceptable diluent or solvent, such as a solution in 1,3-butanediol. Suitable diluents include, for example, water, Ringer's solution and isotonic sodium chloride solution. In addition, sterile fixed oils can be employed conventionally as a solvent or suspending medium. For this purpose, any bland fixed oil can be employed including synthetic mono or diglycerides. In addition, fatty acids such as oleic acid can likewise be used in the preparation of injectable preparations.

It will be appreciated that the amount of the composition may be combined with a carrier material to produce a single dosage form. Such forms will vary depending upon the host treated and the particular mode of administration.

Aqueous suspensions may contain the composition disclosed herein in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

Controlled release vehicles are well known to those of skill in the pharmaceutical sciences. The technology and products in this art are variably referred to as controlled release, sustained release, prolonged action, depot, repository, delayed action, retarded release and timed release; the words “controlled release” as used herein is intended to incorporate each of the foregoing technologies.

Numerous controlled release vehicles are known, including biodegradable or bioerodable polymers such as polylactic acid, polyglycolic acid, and regenerated collagen. Known controlled release drug delivery devices include creams, lotions, tablets, capsules, gels, microspheres, liposomes, ocular inserts, minipumps, and other infusion devices such as pumps and syringes. Implantable or injectable polymer matrices, and transdermal formulations, from which active ingredients are slowly released, are also well known and can be used in the disclosed methods.

Controlled release preparations can be achieved by the use of polymers to form complexes with or absorb the composition. The controlled delivery can be exercised by selecting appropriate macromolecules such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active complex.

Controlled release of active complexes can be taken to mean any of the extended release dosage forms. The following terms may be considered to be substantially equivalent to controlled release, for the purposes of the present disclosure: continuous release, controlled release, delayed release, depot, gradual release, long term release, programmed release, prolonged release, programmed release, proportionate release, protracted release, repository, retard, slow release, spaced release, sustained release, time coat, time release, delayed action, extended action, layered time action, long acting, prolonged action, sustained action medications and extended release, release in terms of pH level in the gut and intestine, breakdown of the molecule and based on the absorption and bioavailability.

Hydrogels, wherein the composition is dissolved in an aqueous constituent to gradually release over time, can be prepared by copolymerization of hydrophilic mono-olefinic monomers such as ethylene glycol methacrylate. Matrix devices, wherein the composition is dispersed in a matrix of carrier material, can be used. The carrier can be porous, non-porous, solid, semi-solid, permeable or impermeable. Alternatively, a device comprising a central reservoir of magnesium picolinate surrounded by a rate controlling membrane can be used to control the release of the complex. Rate controlling membranes include ethylene-vinyl acetate copolymer or butylene terephthalate/polytetramethylene ether terephthalate. Use of silicon rubber depots is also contemplated.

Controlled release oral formulations are also well known. In one embodiment, the composition is incorporated into a soluble or erodible matrix, such as a pill or a lozenge. In another example, the oral formulations can be a liquid used for sublingual administration. These liquid compositions can also be in the form a gel or a paste. Hydrophilic gums, such as hydroxymethylcellulose, are commonly used. A lubricating agent such as magnesium stearate, stearic acid, or calcium stearate can be used to aid in the tableting process.

While the present invention has been described in some detail for purposes of clarity and understanding, one will appreciate that various changes in form and detail can be made without departing from the true scope of the invention.

### EXAMPLES

### Example 1. Comparison of a ZMS Composition with a ZMA Composition

The effects of ZMS and ZMA on visceral fat, biochemical parameters, leptin, anabolic hormones including testosterone and estradiol, serum minerals (magnesium, zinc and selenium), IGF-1, FSH, LH, SHBG, MDA, antioxidant enzymes, absorption of minerals, retina VEGF, iNOS, ICAM Nrf2, bone mineral density, osteocalcin, bone minerals and grip strength are determined. Seven male Wistar rats per treatment arm (age: 8 week, weight: 180±20 g) are housed in a controlled environment with a 12:12-h light-dark cycle at 22° C. and were provided with rat chow and water ad libitum. Following a 7-day acclimatization period, animals are randomly divided into the following groups:
1. Control (no treatment);
2. HFD (fed with high fat diet),
3. HFD+ZMS low dose [MgPic 16.4 mg/day/rat (1.461 mg elemental Mg/day based on converted human dose 100 mg Mg)+ZnPic 2074.6 μg/day/rat (438.28 mg elemental Zn/day based on converted human dose 30 mg Zn)+SeMet 4.222 μg/day/rat (1.46 μg elemental Se/day/rat based on converted human dose 0.1 mg Se)],
4. HFD+ZMS high dose [MgPic 72.63 mg/day/rat (6.574 mg elemental Mg/day based on converted human dose 450 mg Mg)+ZnPic 2074.6 μg/day/rat (438.28 mg elemental Zn/day based on converted human dose 30 mg Zn)+SeMet 4.222 μg/day/rat (1.46 μg elemental Se/day/rat based on converted human dose 0.1 mg Se)];
5. HFD+ZMA [MgAsp, 78.03 mg/day/rat (6.574 mg elemental Mg/day based on converted human dose 450 mg Mg); ZnAsp 1104.25 μg/day/rat and ZnMet 715.65 μg/day/rat (438.28 mg elemental Zn/day based on converted human dose 30 mg Zn), Vitamin B6 (pyridoxal hydrochloride) 153.4 μg/day/rat based on converted human dose 10.5 mg/day]. Half of the elemental zinc is taken from ZnAsp and the other half from ZnMet.

All treatments are administered daily as an oral supplement per day for 8 weeks. The doses above are calculated based on the manufacturer's information that the ZMA supplement contains zinc (30 mg per recommended dose of three capsules, present as monomethionine and aspartate), magnesium (450 mg, as aspartate) and vitamin B6 (10.5 mg, as hydrochloride) (Koehler et al., 2009). To compare the ZMS and ZMA, mineral doses are calculated for rats based on mineral contents of ZMA determined for humans. Metabolic body size is an estimate of the active tissue mass of a person, calculated by the body weight in kilograms to the 0.75 power. Metabolic body weight; body weight 0.75 is generally used to calculate the weight of active tissue. Mg amount is calculated based on 450 mg (high dose) and 100 mg (low dose) that is needed for a 70-kg adult human after adjusting doses based on metabolic body size (70^{0.75}=24.20 kg, needing 450 mg and 100 mg; ˜0.250^{0.75}=0.35 kg needing 6.574 mg/day/rat (Mg) and 1.461 mg/day/rat (Mg). 72.63 mg/day/rat (high dose) and 16.4 mg/day/rat (low dose) MgPic are required as MgPic contains 9.052% elemental Mg. 78.03 mg/day/rat MgAsp is required as MgAsp contains 8.425% elemental Mg.

Zn amount is calculated based on 30 mg that is needed for a 70-kg adult human after adjusting doses based on metabolic body size (70^{0.75}=24.20 kg, needing 30 mg; ˜0.250^{0.75}=0.35 kg needing 438.28 μg (Zn). 2074.6 μg/day/rat ZnPic is required as ZnPic contains 21.126% elemental Zn. 2208.5 μg/day/rat ZnAsp is required as ZnAsp contains 19.845% elemental Zn. 1431.3 μg/day/rat ZnMet is required as ZnMet contains 30.620% elemental Zn.

Se amount is calculated based on 0.1 mg that is needed for a 70-kg adult human after adjusting doses based on metabolic body size (70^{0.75}=24.20 kg, needing 0.1 mg; ˜0.250^{0.75}=0.35 kg needing 0.024 μg/day/rat (Se). 0.068 μg/day/rat SeMet is required as SeMet contains 34.606% elemental Se.

Vitamin B6 (Pyridoxal Hydrochloride), amount is calculated based on 10.5 mg that is needed for a 70-kg adult human after adjusting doses based on metabolic body size (70^{0.75}=24.20 kg, needing 10.5 mg; ˜0.250^{0.75}=0.35 kg needing 153.4 μg/day/rat (Vitamin B6). The average daily consumption of feed for a rat is 15-25 g.

Laboratory analyses—Following the treatment period, plasma is used for the determination of glucose, lipid profile, aspartate transaminase (AST), alanine transaminase (ALT), urea, and creatinine with an automatic analyzer (Samsung). Serum and muscle malondialdehyde (MDA) levels are measured by HPLC (Shimadzu). Total superoxide dismutase (SOD), catalase (CAT) and glutathione peroxidase (GPx) levels are measured using a commercially available assay kit (Cayman Chemical, Ann Arbor, Mich., USA) according to the manufacturer's instructions. Leptin, osteocalcin, testosterone, estradiol, dihydrotestosterone (DHT) and androstenedione, IGF-1, FSH, LH, SHBG are also analyzed by ELISA using a commercially available assay kit.

Atomic Absorption Spectroscopy

All minerals in the sera and tissue samples are measured by flame AAS (Perkin Elmer) using established and fully verified methods (Sahin et al., 2009).

Western Blot Analyses

Brain and retina tissue expression of VEGF, iNOS, ICAM, and Nrf2, and liver expression of SREP-1c, LXR-aACLY, FAS, Nrf2, NFkB are analyzed by Western Blot methods.

Bone Health

The bone mineral density is measured by dual-energy X-ray absorptiometry (Lunar Corp., Madison, Wis., USA). Serum osteocalcin is measured using a commercially available assay kit according to the manufacturer's instructions. Bone mineral density, osteocalcin, and bone minerals are measured according to Sahin et al. (2009) “Effects of 25-hydroxycholecalciferol and soy isoflavones supplementation on bone mineralisation of quail.” Br Poult Sci. 2009 November; 50(6):709-15.

Grip Strength

Grip strength is measured according to Li et al. (2001) Quantitative assessment of forearm muscle size, forelimb gripstrength, forearm bone mineral density, and forearm bone size in determining humerus breaking strength in 10 inbred strains of mice. Calcif Tissue Int. 2001 June; 68(6):365-9. Epub 2001 May 21.

Statistical Analyses

Data is given as mean±SEM. Sample size is calculated based on a power of 85% and a p-value of 0.05. Given that assumption, a sample size of seven per treatment is calculated. The data is analyzed using the GLM procedure of SAS (SAS Institute: SAS User's Guide: Statistics). The treatments are compared using ANOVA and Student's unpaired t-test; P<0.05 is considered statistically significant.

### Example 2. Comparison of a ZMS Composition with a ZMA Composition for Effect on Testosterone

Compositions of the invention were used to test the impact on free testosterone. Compositions of ZMA and ZMS were provided to subjects via supplementation with ZMA and 2 doses of ZMS, which were then compared. Rats, as discussed in Example 1, on a High-Fat Diet (HFD) were assigned to groups accordingly and were administered compositions base on the following: HFD: High-Fat Diet; ZMA: Zinc monomethionine and aspartate (30 mg), Magnesium aspartate (450 mg), and Vitamin B6 as pyridoxine hydrochloride (10.5 mg); ZMS-L (low dose): Zinc picolinate (30 mg), Magnesium picolinate (100 mg) and selenium methionine (100 μg); ZMS: Zinc picolinate (30 mg), Magnesium picolinate (450 mg) and selenium methionine (100 μg).

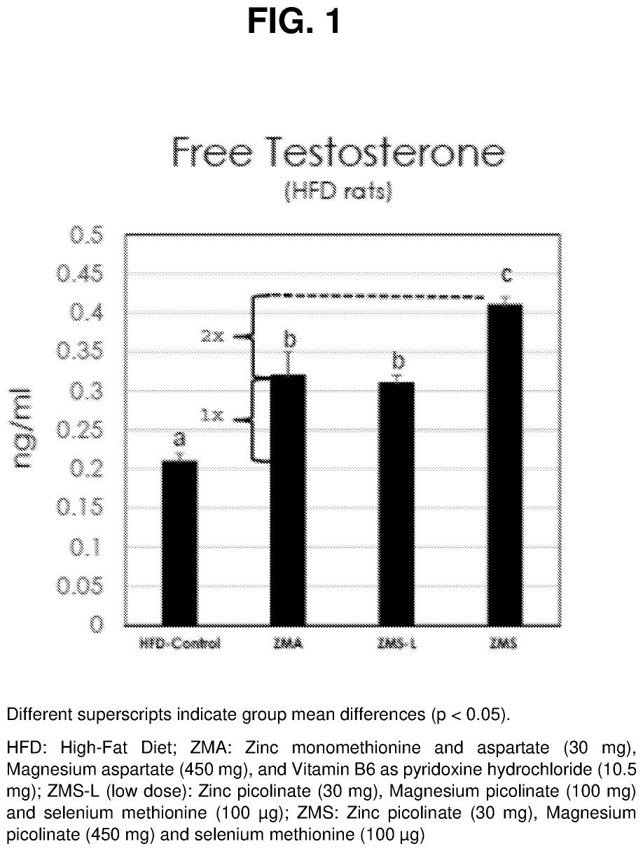

As seen in FIG. 1, the effect of the low Mg dose of ZMS(-L) was unexpectedly similar to that of standard dose levels of ZMA for increasing free testosterone levels. ZMS showed unexpected and superior results when compared to ZMA, with each having the same Mg dose. The effect with ZMS was approximately double that of ZMA at increasing free testosterone levels in the subjects. These results support the superiority of ZMS over ZMA in increasing testosterone.

Improving Sexual Function

Also provided herein are compositions and methods for improving sexual function. Formulations and dosing regimens, as disclosed herein, are applied to subjects in need of improving sexual function. The formulations are personalized depending on the needs of the individual and the function to be improved. The dosing is varied based on the needs of the individual, and based on the methods provided herein.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

When introducing elements of the present application or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

Without further elaboration, it is believed that one skilled in the art can, using the preceding description, utilize the present application to its fullest extent. The specific embodiments are, therefore, to be construed as merely illustrative, and not limitative of the remainder of the disclosure in any way whatsoever. While the present application has been described in some detail for purposes of clarity and understanding, one will appreciate that various changes in form and detail can be made without departing from the true scope of the application.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A composition comprising zinc picolinate, magnesium picolinate and selenium methionine.

2. The composition of claim 1, wherein the composition is a solid composition.

3. The composition of claim 1, wherein the composition comprises a sustained-release matrix.

4. The composition of claim 1, wherein the composition is enteric coated.

5. The composition of claim 1, wherein the composition comprises between about 10 mg to about 10,000 mg of magnesium picolinate, between about 1 mg to about 1000 mg of zinc picolinate, and between about 0.01 mg to about 10 mg of selenium methionine.

6. The composition of claim 1, wherein the composition is used for increasing antioxidant levels in a subject.

7. The composition of claim 1, wherein the composition is used for improving hormone levels.

8. The composition of claim 1, wherein the composition is used for improving bone mineral density.

9. The composition of claim 1, wherein the composition further comprises a pharmaceutically acceptable vehicle, carrier or diluent.

10. A method for improving strength, wherein the method comprises administering to a subject a composition comprising zinc picolinate, magnesium picolinate and selenium methionine in an effective amount to improve strength in the subject.

11. The method of claim 10, wherein the effective amount comprises between about 10 mg to about 10,000 mg of magnesium picolinate, between about 1 mg to about 1000 mg of zinc picolinate, and between about 0.01 mg to about 10 mg of selenium methionine.

12. The method of claim 10, wherein the composition comprises a pharmaceutically acceptable vehicle, carrier or diluent.

13. A method for improving sexual function, wherein the method comprises administering to a subject a composition comprising zinc picolinate, magnesium picolinate and selenium methionine in an amount effective to improve sexual function in the subject.

14. The method of claim 13, wherein the effective amount comprises between about 10 mg to about 10,000 mg of magnesium picolinate, between about 1 mg to about 1000 mg of zinc picolinate, and between about 0.01 mg to about 10 mg of selenium methionine.

15. The method of claim 13, wherein the composition comprises a pharmaceutically acceptable vehicle, carrier or diluent.

16. A method for increasing hormone levels in a subject, where the method comprises administering to a subject a composition comprising zinc picolinate, magnesium picolinate and selenium methionine in an amount effective to increase hormone levels in the subject.

17. A method for increasing antioxidant levels in a subject, where the method comprises administering to a subject a composition comprising zinc picolinate, magnesium picolinate and selenium methionine in an amount effective to increase antioxidant levels in the subject.

18. A method of improving bone density in a subject, where the method comprises administering to the subject a composition comprising zinc picolinate, magnesium picolinate and selenium methionine in an amount effective to improve bone density in the subject.
